(19) United States
(12) Patent Application Publication
(10) Pub. No.: US 2024/0299117 A1
(43) Pub. Date: Sep. 12, 2024

(54) COLLECTION OF HAZARDOUS MATERIALS FOR WASTE MANAGEMENT

(71) Applicant: Whirlwind Intelligent Inspection Systems LLC, Seattle, WA (US)

(72) Inventors: Timothy John WEINERT, Seattle, WA (US); Sahand SABET, Tempe, AZ (US); Sina JAHEDI, Tacoma, WA (US)

(21) Appl. No.: 18/597,905

(22) Filed: Mar. 6, 2024

Related U.S. Application Data
(60) Provisional application No. 63/450,162, filed on Mar. 6, 2023.
(60) Provisional application No. 63/454,553, filed on Mar. 24, 2023.

(51) Int. Cl. A61B 50/36; A61B 90/00; A61B 90/98

(52) U.S. Cl. CPC A61B 50/362; A61B 90/06; A61B 90/98

(57) ABSTRACT

A storage device for collecting hazardous materials is disclosed. In one aspect, the storage device includes a storage container that is configured to store hazardous material. The storage device further includes a collector that is configured to move hazardous material from outside the storage container to inside the storage container. The storage device further includes a sensor that is configured to generate sensor data that reflects a characteristic of an environment in a vicinity of the storage device. The storage device further includes a processor that is configured to analyze the sensor data. The processor is further configured to determine whether to store the item of hazardous material in the storage container. The processor is further configured to determine whether to activate the collector to move the item of hazardous material from outside the storage container to inside the storage container.

### CROSS-REFERENCE TO RELATED APPLICATIONS

This application claims the benefit of U.S. Application 63/450,162, filed Mar. 6, 2023, and U.S. Application No. 63/454,553, filed Mar. 24, 2023, which are both incorporated by reference.

### BACKGROUND

Hazardous materials such as syringes, dangerous chemicals, biological materials, oil, fuels, and the like may pose a serious risk to the public and/or local environment when not disposed of properly. Detecting these hazardous materials may range from manually scanning an open field to, for example, using detecting devices that utilize device sensors. In recent times, more technologies are being developed to improve the detection of these hazardous materials.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 is a diagram of an example network server environment, in accordance with at least one embodiment.

FIG. 2 is a diagram of an example detecting device, in accordance with at least one embodiment.

FIG. 3 is a flow diagram of an example procedure, in accordance with at least one embodiment.

FIG. 4 is a flow diagram of another example procedure, in accordance with at least one embodiment.

FIG. 5 is a flow diagram of yet another example procedure, in accordance with at least one embodiment.

### DETAILED DESCRIPTION

This disclosure describes systems and methods for improving collection of hazardous materials and post-collection actions performed by the devices collecting the hazardous materials. Particularly, the devices collecting the hazardous materials may use various techniques to identify the type of hazardous material, the integrity of the hazardous material, and the risk posed to the surrounding environment and nearby people by the hazardous material. The devices may analyze sensor data collected by the device as part of these various techniques. After collection of the hazardous material, the collecting device may determine its next movements. The movements may be based on the type of hazardous material, the integrity of the hazardous material, the risk posed to the surrounding environment and nearby people by the hazardous material, and/or the sensor data. Hazardous materials may include, without limitation, syringes, painting wastes, bio-waste, dangerous chemicals, litter, trash, and other types of objects that can provide risks to persons or local environment. Although the embodiments herein focused on syringes, these embodiments may similarly apply to other types of hazardous materials.

In one embodiment, the detecting device may use various syringe identification models and rules to analyze sensor data collected by the detecting device. Based on the outputs of the syringe identification models and rules, the detecting device may determine when it is in the vicinity of a syringe and the location of that syringe. With the location of the syringe determined, the detecting device may further determine a type of the syringe. The type of syringe may be related to the material used to manufacture the syringe. The materials may include cyclo-olefin-polymer, cyclo-olefin-copolymer, zylar, borosilicate glass, polyethylene, polypropylene, metal, and/or any other similar material. The type of syringe may be related to the number of parts that make up the syringe, such as a two or three part syringe. The type of syringe may be a luer slip, luer lock, eccentric, centric, catheter tip, oral tip, or any other type of syringe. The type of syringe may also include whether the syringe includes a needle.

The detecting device may further determine the integrity of the syringe. The integrity of the syringe may include whether the syringe is intact or broken. Broken syringes may include those that are crushed by things such as a tire or shoe. The integrity of syringes may also include whether any part of the syringe has decomposed.

The detecting device may further estimate the risk that the syringe poses to the environment and/or to nearby people. The risk may be on a scale of zero to one, with zero being no risk and one being immediate and serious risk.

Based on the type of syringe, the integrity of the syringe, and the risk of the syringe, the device may determine a collection technique. The collection technique may include collecting the syringe using a scoop, an arm, vacuum system and/or any other similar collection technique. In some implementations, the collection technique may involve the device covering the syringe and becoming immobile such that it may be difficult for a person to move. The collection technique may include where to store the syringe. In some implementations, the device may include multiple collection bins. Some of the bins may be puncture resistant. These may be constructed of a sturdier and heavier material that prevent needles from compromising the collection bin. Other bins may not be puncture resistant. These may be constructed of lighter, less-sturdier material. These bins may be punctured by needles. The bins themselves may contain various container sensors to count the objects collected, weighting the objects, altering the RFID tag data on the collected objects, and so on.

Once the collecting device collects the syringe, the collecting device should determine where to move next. In some implementations, the collecting device may move towards the likely location of additional syringes. In some implementations, the collecting device may determine to move to a different location. The different location may include a home base, the location of an additional collecting device, or not move anywhere. The collecting device may make this determination based on various factors. The factors may include the sensor data collected, the capacity of the collecting bins, the remaining battery capacity of the collecting device, the capacity of collection bins of other nearby collecting devices, the remaining battery capacity of the nearby collecting devices, the likely locations of additional syringes, the likelihood that those locations will have syringes, the location of the home base, and/or any other similar factors. In some implementations, the factors may also include the type of syringes, the integrity of the syringes, and the risk of the syringes in the collection bins.

In some implementations, the syringe identification models may be derived from historical data of previously identified syringes and their corresponding associated data. The associated data may include, for example, sensor data and environmental data that were used to identify the syringes. In some cases, syringe identification rules that can include pre-defined or user-defined rules may be used to identify the syringes. For example, the syringe identification rules may use a threshold for a received strength of received radio-frequency identification (RFID) signals to determine how close is the syringe. In this example, the threshold may be pre-defined.

In one embodiment, a server, such as a waste management operating center (WMOC) server, may utilize the syringe identification models on received sensor data to identify whether the received sensor data is associated with a syringe. The sensor data may be captured by a detecting device such as, without limitation, an unmanned aerial vehicle (UAV) or known as drone, a mobile device, ground vehicle, hand-head device, a camera, light detection and ranging (LIDAR), a ultraviolet (UV) light detector, metal detector, fluorescent detector, chemical detector, an Internet of Things (IoT) device, or a combination thereof. The received sensor data may include, without limitation, RFID signals, optical tags, captured physical shapes, natural light reflections, heat radiations, chemical odor, or a combination thereof. The detected optical tags may include identified bar codes, QR codes, ultraviolet (UV) rays, printed fluorescent dyes, and the like. In this embodiment, a portion or all of the received sensor data may be used as inputs to the syringe identification models to generate an output that can include the identification of the object as syringe.

In some cases, and upon receiving of the sensor data, the WMOC server may receive from third-party sources or servers the environmental data that can be associated with a geolocation of the detecting device. The environmental data may include a nature or context that can be associated with the geolocation of the detecting device. For example, the nature may include a community street that can be associated with a low population density, a city street associated with historically high crime rate, hospital area where syringes may be regularly found, a crop field where there is low probability of finding syringes, or other area where chances of finding the syringes may be associated with classification or type of the area.

The context may include presence of a recent activity such as a music concert, day of the week that can be associated with historically increased number of dumped syringes, real-time events that are proximate to the geolocation of the detecting device, and the like. In some embodiments, the context may also include data from third-party server(s), news reports, social media postings, or reports that can describe the disposition of a surrounding environment proximate to the geolocation of the detecting device.

While the sensor data alone may provide a good prediction for the identification of the syringes in ideal detecting conditions such as clear sky, less signal noise interference, etc., the addition of the environmental data can improve a likelihood of the identification and by extension, the estimation of the geolocation of the syringes.

For example, current weather conditions (environmental data) may affect the detection of the wireless signals, capturing of optical tags, or scanning of other identification features such as preconfigured shapes of the hazardous materials. In this example, the current weather conditions, in addition to the captured sensor data, may be used as input data to the syringe identification models to identify the syringe and by extension, the geolocation of the identified hazardous material. In some cases, the syringe location estimation models may be used independently on captured sensor data and environmental data to generate a likely geolocation of the additional syringes. For example, the environmental data includes a recently performed adult concert in a nearby area. In this example, the syringe location estimation models may output a likely location of syringes based on receiving data indicating the concert occurred in the nearby area.

Upon identification of the syringes and the estimation of their corresponding geolocations, one or more ML algorithms may be further employed to infer an ordered ranking of priority for collecting the syringes at their corresponding identified geolocations. In one embodiment, a syringe collection model may be utilized on the identified geolocations of the syringes and the environmental data associated with the identified geolocations to infer the ordered ranking of priority for the collection of these syringes.

For example, the identified geolocations of the syringes to be collected may include a children's playground, dumpsite, and a medical clinic. Here, the environmental data that is associated with each of these identified geolocations may be used as an additional input in the syringe collection model for inferring the ordered ranking of priority for the collection of the syringes. For example, the syringes at the children's playground may be given priority for collection compared to the syringes that are geolocated at the dumpsite. In another example, the syringes at the medical clinic may be given priority for collection compared to the syringes that are geolocated at the dumpsite, and so on.

In some embodiments, the inferred order of ranking for priority of collection of the syringes may be used as a reference to create a dispatch schedule for one or more waste collectors that are assigned to collect these hazardous materials. For example, a waste management service provider employs multiple waste collectors that are randomly scattered in different locations. In this example, a linear algorithm may be used to create the dispatch schedule to maximize availability of the waste collectors, maximize profits for the service provider, or a combination of both.

Further, the term “techniques,” as used herein, may refer to system(s), method(s), computer-readable instruction(s), module(s), algorithms, hardware logic, and/or operation(s) as permitted by the context described above and through the document.

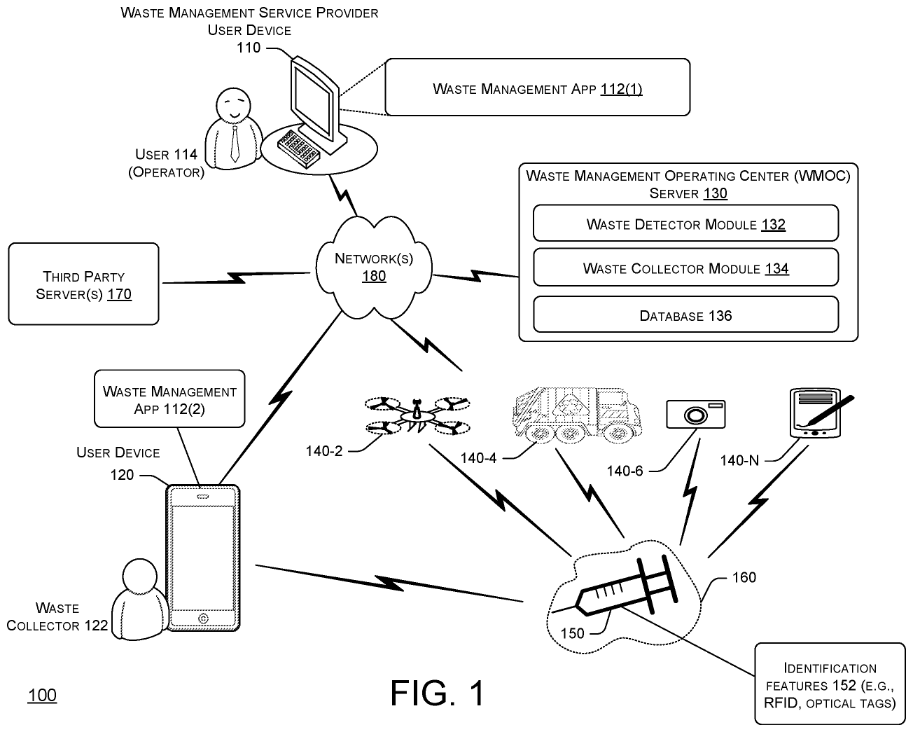

FIG. 1 illustrates a schematic view of a network server environment 100 that facilitates an identification of hazardous materials and estimation of geolocations of additional hazardous materials for collection and proper disposal by waste management personnel. Hazardous items may include, without limitation, syringes, painting wastes, bio-wastes and other type of dangerous goods that can be potential threats to public health or environment. In one embodiment, a waste management operating center (WMOC) server may support subscribers of a waste management application (app) for the identification, estimation of geolocation, and collection of these hazardous materials. The waste management app may be installed in user devices associated with waste management service provider, detecting devices, or user devices associated with service provider personnel. In this embodiment, the waste management app may improve the identification of the hazardous materials, detection of geolocations of additional hazardous materials, and collection of the identified hazardous materials. Although the main embodiment as described herein utilizes the syringes as the type of hazardous materials to be identified, geolocated, and/or collected, the embodiments may similarly apply to the other types of hazardous materials such as paint, lead metals, aerosols, and the like.

As shown, the network environment 100 may include a waste management service provider user device 110 with an installed waste management app 112(1), a user 114 such as a waste management operator or manager, a user device 120 with an installed waste management app 112(2), a waste collector 122 who is associated with the user device 120, a network server such as a WMOC server 130, detecting devices 140-2, 140-4, . . . 140-N (detecting devices 140), a syringe 150 that is located at a geolocation point 160, third party server(s) 170, and one or more networks 180. The WMOC server 130 may further include a waste detector module 132, waste collector module 134, and a database 136. The syringe 150 may further include identification features 152 such as, without limitation, RFID tag, optical tags, syringe shape, fluorescent dye, or other detectable features that can be captured by the detecting devices 140 and/or user device 120. In some embodiments, the network environment 100 may be or include a cellular network.

The waste management service provider user device 110 or the user device 120 may include an electronic communication device, including but not limited to, a smartphone, a session initiation protocol (SIP) phone, a laptop, a personal digital assistant (PDA), a satellite radio, a global positioning system (GPS), a multimedia device, a video device, a camera, a LIDAR, a game console, a tablet, a smart device, a wearable device, or any other similar functioning device. In one embodiment, the service provider user device 110 or the user device 120 may use the waste management app 112(1) or 112(2) to communicate with the WMOC server 130. The waste management app 112(1) or 112(2) (also referred to as waste management app 112) may be used to transmit data to the web server 130 and/or receive data such as the identified geolocations of the syringes and ordered ranking of priority to collect the syringes at the identified geolocations.

The WMOC server 130 may utilize distributed computing resources (e.g., one or more computing devices) that can operate in a cluster or other configuration to share resources, balance load, increase performance, provide fail-over support or redundancy, or for other purposes. The WMOC server 130 may include one or more interfaces to enable communications with the service provider user device 110, user device 120, detecting devices 140, and other networked devices via the one or more network(s) 180. In one embodiment, the WMOC server 130 may receive sensor data that can be captured by the user device 120 and/or the detecting devices 140. The sensor data may include, without limitation, RFID signals, detected UV fluorescent rays, detected shape, or a combination thereof. In this embodiment, the WMOC server 130 may use prediction models to identify the syringe 150 and further to estimate the geolocation 160 of additional syringes. The detecting devices 140 may determine how to collect the syringes and what action to perform after collection of the syringes.

The WMOC server 130 may also receive environmental data from the third party server(s) 170. The environmental data may include the data that can be associated with the geolocation of the detecting device. In some implementations, the environmental data may include the nature or context that can be associated with a monitored geolocation of the detecting device. The nature may include, without limitation, a community street that can be associated with a low population density, a city street associated with historically high crime rate, hospital area where syringes may be regularly found, a crop field where there is low probability of finding syringes, or other area where chances of finding the syringes may be associated with type or classification of the area.

The context may include presence of a recent activity such as a music concert, day of the week that can be associated with historically increased number of dumped syringes, real-time events that are proximate to the geolocation of the detecting device, and the like. In some embodiments, the context may also include data from third-party server(s), news reports, social media postings, or reports that describe the disposition of a surrounding environment proximate to the geolocation of the detecting device.

With the collected sensor data and environmental data, the WMOC server 130 may use the waste detector module 132 to identify whether an object is a syringe or not. In some embodiments, the waste detector module 132 may use syringe identification models on the collected sensor data and environmental data to identify the syringe 150. Further, the waste detector module 132 may determine the type, integrity, and risk level of the syringes.

In one implementation, the WMOC server 130 may use the waste collector module 134 to infer an ordered ranking of priority for collecting the identified syringes at their corresponding estimated geolocations. For example, a syringe collection model may be trained from the historical geolocations of the collected syringes and risks associated with the historical geolocations. In some embodiments, an inferred order of ranking for priority of collection of the syringes may be used as a reference by the WMOC server 130 to create a dispatch schedule for one or more waste collectors that are assigned to collect these hazardous materials. For example, a waste management service provider employs multiple waste collectors such as the waste collector 122. In this example, a linear algorithm may be used to create the dispatch schedule to maximize availability of the waste collectors, maximize profits for the service provider, or a combination of both.

The waste management app 112 may include an application that can be installed in a device such as the user device 120 or the service provider user device 110 to access the WMOC server 130. In one embodiment, the user device 120 or the service provider 110 may be required to enter information when subscribing to the waste management app 112. The information may include, without limitation, identification of person associated with the user device 120, identification of the service provider associated with the provider device 110, and other device data. This information may be stored in the database 136 of the WMOC server 130.

The user 114 may include an individual who can be a waste management service provider owner, manager, or operator. In one embodiment, the user 114 may use the service provider user device 110 and particularly, the waste management app 112(1) to access the WMOC server 130. For example, the service provider user device 110 may access and collect the data of the geolocations of the syringes and the created dispatch schedule (not shown). In this example, the service provider user device 110 may communicate with the user device 120 and/or detecting devices 140 to implement the created dispatch schedule.

The waste collector 122 may include an individual who can be an employee of the waste management service provider, or hobbyist who is a subscriber of the waste management app 112 just to collect hazardous materials. In one embodiment, the waste collector 122, via the user device 120, may receive instructions or the dispatch schedule using the installed waste management app 112(2).

The one or more network(s) 180 may include public networks such as the Internet, private networks such as an institutional and/or personal intranet, or some combination of private and public networks. The one or more network(s) 180 can also include any type of wired and/or wireless network, including but not limited to local area network (LANs), wide area networks (WANs), satellite networks, cable networks, Wi-Fi networks, Wi-Max networks, mobile communications networks (e.g., 3G, 4G, and so forth), or any combination thereof.

The detecting devices 140 may include an unmanned aerial vehicle (UAV) or known as drone, a mobile device, metal detector, chemical detector, pressure detector, fluorescent detector, an Internet of Things (IoT) device, a UV light detector or any other types of sensor devices. In one embodiment, the detecting device 140-2 is a drone that hovers above ground and detects the identification features 152 associated with the syringe 150. In another embodiment, the detecting device 140-4 is a garbage truck that uses metal detectors or RFID sensors to detect the identification features 152. In another embodiment, the detecting device 140-6 is a camera, and so on. Without limitation, the identification features 152 may include RFID signals that can be received at different points, optical tags, preconfigured physical shape, preconfigured location of the tags in the body of the syringe, or a combination thereof. Here, while the RFID signals may be used in a triangulation method to detect the geolocation 160 of the syringe 150, the additional and/or alternative use of the other identification features 152 may further improve the detection of the geolocation 160 of the syringe 150.

For example, during a rainy weather condition, the use of the RFID signals or other short range communications may be affected by the rain and thus, the optical tags or detected shape may be added to improve the detection of the geolocation 160. In another example, in a covered area where the optical tags are not visible to the detecting devices 140, the RFID signal may be used to identify the geolocation 160. In these examples, the sensor data associated with the collected syringes may be used as historical data to train the prediction model as described herein.

In some embodiments, the detecting devices 140 may include a combination of sensors in the garbage truck 140-4, fluorescent detector in the camera 140-6, and so on. Here, the WMOC server 130 may collect sensor data from these detecting devices 140.

The syringe 150 may include a reciprocating pump with a plunger that fits within cylindrical tube or barrel of the pump. In one embodiment, the cylindrical tube may be associated with a RFID tag, optical tags, preconfigured shape, or a combination thereof. The tags can be added to the cylindrical tube using adhesive, sticker tapes, or such. Also, the tags can be embodied into the plunger or cylindrical tube of the syringe. The same with fluorescent dyes, it can be added to a sticker that wraps around the plunger or cylindrical tube, premixed with the plastic during fabrication of the syringe, or painted on inner or outer body of the syringe after fabrication. In some embodiments, the optical tags or RFID device may be used as a reference for another identification feature of the syringe 150. For example, a detection of the RFID signal may indicate presence of an optical tag that can be used to identify the syringe 150. In another example, a capturing of the optical tag may indicate associated preconfigured shape or size of the syringe. In another example, a detection of a particular RFID signal may indicate absence of additional optical tags to identify the syringe, and so on.

In an example operation, the WMOC server 130 may receive a request from the user device 110 to identify the syringes over a particular area. Here, the WMOC server 130 may receive captured sensor data from the detecting devices 140 and/or the user device 120. The WMOC server 130 may also receive the environmental data from the third party server(s) 170. The WMOC server 130 may then use the prediction models in the waste detector module 132 and/or the waste collector module 134 to identify the syringe 150, estimate the geolocation 160 of syringes other than the syringe 150, determine the integrity of the syringe 150, determine the risk level of the syringe 150, determine the type of the syringe 150, and/or rank the order of priority for the collection of the identified syringes at their corresponding geolocations.

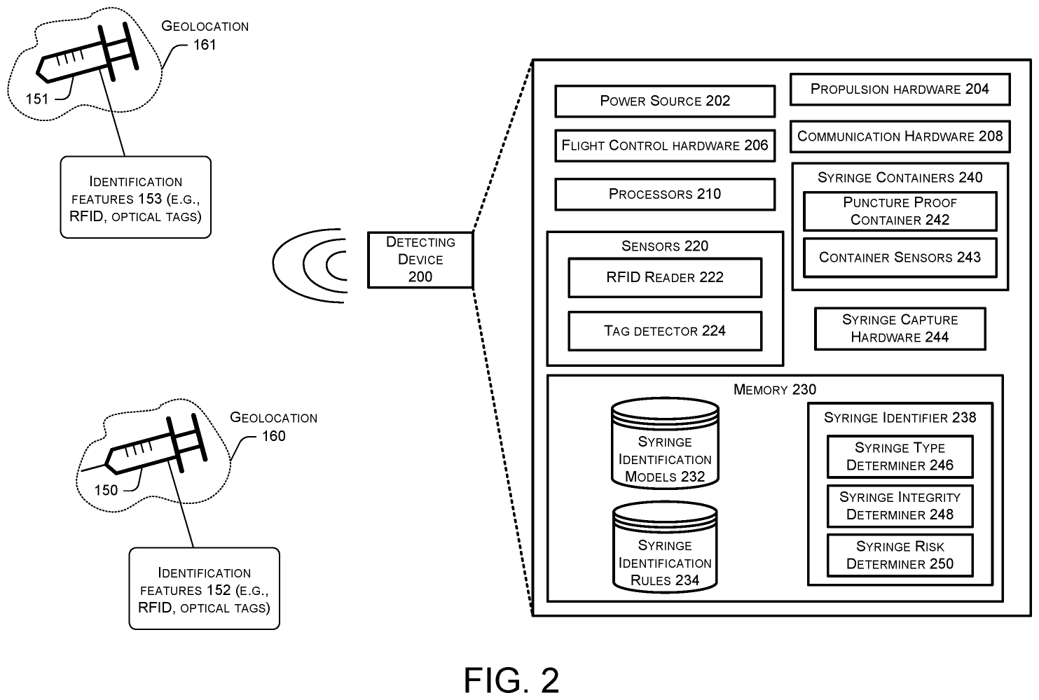

FIG. 2 is a block diagram showing various components of a detecting device 200 that is configured to identify syringes, determine a method of collection of syringes, and determine how to proceed after collection of syringes. In some implementations, the detecting device 200 is a drone that can utilize the syringe identifier 238 to the syringes 150 and 151 and determine their types, integrity, and risk level.

As shown, the detecting device 200 may include a power source 202, propulsion hardware 204, flight control hardware 206, communication hardware 208, one or more processors 210, sensors 220, syringe containers 240, syringe capture hardware 244, and memory 230. The sensors 220 may include, without limitation, an RFID reader 222, a camera, a LIDAR device or detector, a tag detector 224, and/or any other similar sensor. The memory 230 may include syringe identification models 232, syringe identification rules 234, and syringe identifier 238. The syringe container 240 may include a puncture proof container 242. The syringe identifier 238 may include various subcomponents such as a syringe type determiner 246, a syringe integrity determiner 248, and a syringe risk determiner 250. In some implementations, the syringe identification models 232 and the syringe identification rules 234 may be trained on another device, such as a WMOC server. The WMOC server may provide the syringe identification models 232 and the syringe identification rules 234 to the detecting device 200, along with periodic updates to the syringe identification models 232 and the syringe identification rules 234.

The power source 202 may include electrical cells, combustible liquid fuel, combustible gas fuel, compressed gas, and/or other energy sources. In some embodiments, the power source 202 may be a ground-based energy source, rather than an energy source that is carried onboard the detecting device 200. In such embodiments, a power line or fuel line may convey the energy from the ground-based energy source to the detecting device 200. The propulsion hardware 204 may include mechanical devices that can convert the energy provided by the power source 202 into movements of the detecting device 200. For example, the propulsion hardware may include an internal combustion engine, an electrical motor, a jet engine, a turboprop engine, propellers, rotors, and/or so forth that are mounted on the wings and/or the body of the detecting device 200.

The flight control hardware 206 may include actuators and control surfaces that can steer the detecting device 200. For example, the actuators may include hydraulic actuators, gas-powered actuators, electrical actuators, and/or so forth. The actuators may move or deflect control surfaces to control the movement of the detecting device 200. The control surfaces may include tilt wings, rudders, slats, ailerons, elevators, trim tabs, fins, canards, and/or so forth. In some embodiments, the flight control hardware 206 may be integrated with the propulsion hardware 204. For example, such integrated hardware may include tilt rotors, variable pitch rotors, jet engines with movable thrust nozzles, and/or so forth.

The communication hardware 208 may include hardware components that enable the detecting device 200 to communicate with other detecting devices, a WMOC server, devices associated with the waste collectors, or devices associated with the waste management service provider. In various embodiments, the communication hardware 208 further includes cellular transceivers, hardware decoders and encoders, an antenna controller, a memory buffer, a network interface controller, a universal serial bus (USB) controller, and/or other signal processing and communication components.

Accordingly, the communication hardware 208 may support the transmission and reception data for wireless communication. The communication hardware 208 may further include one or more antennas that support the transmission and reception of data signals. The antennas may include a Yagi antenna, a horn antenna, a dish reflector antenna, a slot antenna, a waveguide antenna, a Vivaldi antenna, a helix antenna, a planar antenna, a dipole array antenna, an origami antenna, and/or other types of antennas. In some instances, an antenna may be oriented to point to a direction via electrical beamforming and/or via mechanical movement of one or more elements of the antenna by an antenna controller.

The sensors 220 may be configured to capture sensor data from objects such as the syringes 150 and 151 and/or any objects in the vicinity of the detecting device 200. The RFID reader 222 may detect RFID signals while the tag detector 224 may capture fluorescent tags, bar codes, and the like. Other sensors 220 may include a camera, a LIDAR image sensor, shape detector, metal detector, optical sensor, chemical sensor, proximity sensor, light sensor, IR sensor, accelerometer, temperature sensor, an altitude sensor, a global positioning system (GPS) sensor, control setting sensors, three-dimensional scanning device (e.g., LIDAR, stereo camera, etc.), propulsion setting sensors, and the like. In some implementations, the sensors 220 may coordinate with other devices. The other devices may include a light source, an ultraviolet light source, an infrared light source, and/or any other similar type of device that is configured to provide an output to improve the accuracy of the sensor data. For example, the sensor data may be collected while illuminating the area with visible light and UV light either simultaneously or in sequence. In some implementations, the sensors 220 may include duplicate sensors. For example, two image sensors to improve depth perception. The sensors 220 may provide the sensor data, via the communication hardware 208, to a WMOC server for further processing and/or may provide the sensor data to the processors 210.

Each of the processors 210 may be a single-core processor, a multi-core processor, a complex instruction set computing (CISC) processor, or another type of processor. The memory 230 may be implemented using computer-readable media, such as computer storage media. Computer-readable media includes, at least, two types of computer-readable media, namely computer storage media and communications media. Computer storage media includes volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital storage disks or other optical storage, magnetic cassettes, magnetic tape, magnetic disk storage or other magnetic storage devices, or any other non-transmission medium that can be used to store information for access by a computing device. In contrast, communication media may embody computer-readable instructions, data structures, program modules, or other data in a modulated data signal, such as a carrier wave, or other transmission mechanisms.

The syringe containers 240 may include one or more types of containers that are configured to store collected syringes. The syringe containers 240 may include a puncture proof container 242 that is configured to store syringes. Other syringe containers 240 may not be puncture proof. Different types of containers may be made of different materials. Some materials may have different densities, costs, availability, flexibility, malleability, ductility, ability to be cleaned, and/or any other similar differences. In some implementations, the material of the puncture proof container 242 may be different than the material for a non-puncture proof container. The puncture proof container 242 may have a higher cost and density than the material for a non-puncture proof container. Accordingly, the syringe containers 240 may include multiple containers such as a puncture proof container 242 that is smaller than other containers such as a non-puncture proof container.

In some implementations, the syringe containers 240 may include additional container sensors 243. The container sensors 243 may include sensors that are configured to count the number of syringes in the syringe containers 240, weigh each of the syringes in the syringe containers 240, alter RFID tag data on the collected objects, and/or any other similar type of sensor.

The syringe capture hardware 244 may include various implements used to collect and place the syringes in the syringe containers 240. The syringe capture hardware 244 may include scoops, a robotic arm, claws, vacuum systems, and/or other implements that are configured to pick up syringes. In one example, the syringe capture hardware may use a wire ball to collect the syringes as it rolls over the object. In another example, the capture hardware may use magnets. Some of the syringe capture hardware 244 may be configured to collect syringes that may be crushed. Some of the syringe capture hardware 244 may be configured to collect syringes that may include sharp objects, such as needles. Some of the syringe capture hardware 244 may be configured to collect syringes that may include hazardous materials. In some implementations, the syringe capture hardware 244 may include materials that may neutralize any hazardous materials. These materials may be placed on the location of the syringe before and/or after collecting the syringe. In some implementations, the syringe capture hardware 244 may include objects that are configured to be left at the site of the syringe. These objects may include protection domes that may cover the location of the syringe. In some implementations, the syringe may remain, which may occur if the syringe containers 240 do not have space to store the syringe.

The memory 230 may store software code for the syringe identification models 232 and the syringe identification rules 234. The memory 230 may also include software code that allows the processors 210 to implement a syringe identifier 238. The syringe identifier 238 may use the syringe identification models 232 and the syringe identification rules 234 to analyze sensor data and any additional data to determine when the detecting device 200 is in the vicinity of a syringe. Due to different types of sensors used and thus, different sensor data that are captured by different detecting devices, each detecting device may be associated with corresponding syringe identification models 232 and syringe identification rules 234. For example, the configured syringe identification model for a drone detecting device is different from the configured syringe identification model for a chemical detector. In this example, the sensor data that can be captured by the drone detecting device is different from the sensor data that can be captured by the chemical detector. In this regard, the historical sensor data that can be used to train these detecting devices may be different. The resulting models may be configured to receive the same type of data used to train them.

In one embodiment, the syringe identification models 232 that can be used by the syringe identifier 238 to determine whether an object is a syringe or not. In this embodiment, the syringe identifier 238 may use captured sensor data as input, for example, to the syringe identification models 264 to determine whether the captured sensor data corresponds to a detected syringe. In some implementations, the syringe identifier 238 may use the syringe identification rules 234 to analyze the sensor data. The syringe identification rules 234 may include various thresholds, ranges, and/or other comparison techniques to determine whether the sensor data indicates that a syringe is likely in the vicinity of the detecting device 200.

The processors 210 may implement a syringe type determiner 246, a syringe integrity determiner 248, and a syringe risk determiner 250. In some implementations, the syringe identification models 232 and the syringe identification rules 234 may include rules and models that each respective component of the syringe identifier 238 may use to make their respective determination. The syringe type determiner 246 may access syringe identification models 232 and the syringe identification rules 234 that are configured to receive sensor data, context data, data indicating the likely location of a syringe, and/or any other similar data. The syringe type determiner 246 may output data indicating the likely type of the syringe. The type of syringe may be related to the material used to manufacture the syringe. The materials may include cyclo-olefin-polymer, cyclo-olefin-copolymer, zylar, borosilicate glass, polyethylene, polypropylene, metal, and/or any other similar material. The type of syringe may be related to the number of parts that make up the syringe, such as a two or three part syringe. The type of syringe may be a luer slip, luer lock, eccentric, centric, catheter tip, oral tip, or any other type of syringe. The type of syringe may also include whether the syringe includes a needle.

For example, the syringe type determiner 246 may receive data indicating that the detecting device 200 is likely in the vicinity of the syringe 150. The syringe type determiner 246 may use the syringe identification models 232 and the syringe identification rules 234 to analyze the sensor data, context data, data indicating the likely location of a syringe, and/or any other similar data. The syringe type determiner 246 may determine that the syringe 150 includes a needle, is made of plastic, and is a two-part syringe.

As another example, the syringe type determiner 246 may receive data indicating that the detecting device 200 is likely in the vicinity of the syringe 151. The syringe type determiner 246 may use the syringe identification models 232 and the syringe identification rules 234 to analyze the sensor data, context data, data indicating the likely location of a syringe, and/or any other similar data. The syringe type determiner 246 may determine that the syringe 151 does not include a needle, is made of glass, and is a two-part syringe.

The syringe integrity determiner 248 may access syringe identification models 232 and the syringe identification rules 234 that are configured to receive sensor data, context data, data indicating the likely location of a syringe, data indicating the type or syringe, and/or any other similar data. The syringe integrity determiner 246 may output data indicating the likely integrity of the syringe. The integrity of the syringe may include whether the syringe is intact or broken. Broken syringes may include those that are crushed by things such as a tire or shoe. The integrity of syringes may also include whether any part of the syringe has decomposed.

For example, the syringe integrity determiner 248 may receive data indicating that the syringe type identifier 238 has determines that the syringe 150 includes a needle, is made of plastic, and is a two-part syringe. The syringe integrity determiner 248 may use the syringe identification models 232 and the syringe identification rules 234 to analyze the sensor data; context data; data indicating the syringe 150 includes a needle, is made of plastic, and is a two-part syringe; and/or any other similar data. The syringe integrity determiner 248 may determine that the syringe 150 is intact and has not started to decompose.

For example, the syringe integrity determiner 248 may receive data indicating that the syringe type identifier 238 has determines that the syringe 151 does not include a needle, is made of glass, and is a two-part syringe. The syringe integrity determiner 248 may use the syringe identification models 232 and the syringe identification rules 234 to analyze the sensor data; context data; data indicating the syringe 151 does not include a needle, is made of glass, and is a two-part syringe; and/or any other similar data. The syringe integrity determiner 248 may determine that the syringe 151 intact and is ten percent decomposed.

The syringe risk determiner 250 may access syringe identification models 232 and the syringe identification rules 234 that are configured to receive sensor data, context data, data indicating the likely location of a syringe, data indicating the type or syringe, data indicating the integrity of the syringe, and/or any other similar data. The syringe risk determiner 250 may output data indicating the likely risk of the syringe. The risk of the syringe may estimate the risk that the syringe poses to the environment and/or to nearby people. The risk may be on a scale of zero to one, with zero being no risk and one being immediate and serious risk.

For example, the syringe risk determiner 250 may receive data indicating that the syringe type identifier 238 has determined that the syringe 150 includes a needle, is made of plastic, and is a two-part syringe and data indicating that the syringe integrity determiner 248 indicated that the syringe 150 is intact and not decomposed. The syringe risk determiner 250 may use the syringe identification models 232 and the syringe identification rules 234 to analyze the sensor data; context data; data indicating the syringe 150 includes a needle, is made of plastic, and is a two-part syringe; data indicating that the syringe 150 is intact and not decomposed; and/or any other similar data. The syringe risk determiner 250 may determine that the syringe 150 has a risk score of 0.6.

For example, the syringe risk determiner 250 may receive data indicating that the syringe type identifier 238 has determined that the syringe 150 does not include a needle, is made of glass, and is a two-part syringe and data indicating that the syringe integrity determiner 248 indicated that the syringe 151 is intact and ten percent decomposed. The syringe risk determiner 250 may use the syringe identification models 232 and the syringe identification rules 234 to analyze the sensor data; context data; data indicating the syringe 151 does not include a needle, is made of glass, and is a two-part syringe; data indicating that the syringe 151 is intact and ten percent decomposed; and/or any other similar data. The syringe risk determiner 250 may determine that the syringe 150 has a risk score of 0.3.

Based on the determinations from the syringe type identifier 238 syringe integrity determiner 248, and syringe risk determiner 250, the processors 210 may determine how to capture the syringes 150 and 151. The processors 210 may select a component of the syringe capture hardware 244. The components of the syringe capture hardware 244 may include a claw, a scoop, grabber arm, a rake, a broom, a vacuum, a magnet, and/or any other similar implements that may be used to manipulate and/or collect a syringe. The processors 210 may select the component and activate that component. The syringe identifier 238 may continue to monitor the location of the syringe to determine whether the syringe has been collected. If the syringe has not been collected, then the processors 210 may select a different component of the syringe capture hardware 244 to attempt to capture the syringe. If the syringe has been collected, then the processors 210 may determine where to store the collected syringe.

If the syringe continues to remain either in whole or in part after the processors 210 have selected each of the implements of the syringe capture hardware 244, then the processors 210 may utilize a portion of the syringe capture hardware 244 that may be designed to remain at the location of the syringe. These portions of the syringe capture hardware 244 may include domes, boxes, and/or any other similar object that is designed to cover another object.

The processors 210 may determine where to store the collected syringe. The syringe containers 240 may include various containers that are configured to store syringes. The syringe containers 240 may include a puncture proof container 242 that is configured to store syringes that may include needles without allowing the needles to puncture the puncture proof container 242. The syringe containers 240 may include additional containers that may not be puncture proof. The syringe containers 240 may be made of materials that have different densities, costs, availability, flexibility, malleability, ductility, ability to be cleaned, and/or any other similar differences. Each of the syringe containers 240 may have a different capacity. The processors 210 may select one of the syringe containers 240 for storing the collected syringe based on the characteristics of the collected syringe. The characteristics may include the type, the integrity, the risk posed by the syringe, and/or any other similar characteristic.

As an example, the processors 210 may receive data related to the syringe 150. That data may include that the syringe 150 includes a needle, is made of plastic, and is a two-part syringe; is intact and not decomposed; and has a risk score of 0.6. The processors 210 may also receive sensor data of the vicinity of the syringe 150. Based on analyzing this data, the processors 210 may select a grabber arm of the syringe capture hardware 244 to collect the syringe 150. If successful and based on a portion or all of the same data, the processors 210 may determine to store the syringe 150 in the puncture proof container 242.

As another example, the processors 210 may receive data related to the syringe 151. That data may include that the syringe 151 does not include a needle, is made of glass, and is a two-part syringe; is intact and ten percent decomposed; and has a risk score of 0.3. The processors 210 may also receive sensor data of the vicinity of the syringe 151. Based on analyzing this data, the processors 210 may select a scooper of the syringe capture hardware 244 to collect the syringe 150. If successful and based on a portion or all of the same data, the processors 210 may determine to store the syringe 150 in one of the syringe containers 240 other than the puncture proof container 242.

At some point during the collection of syringes, the processors 210 may make a determination to cease collection of syringes and take another action. The determination may be based on various factors. These factors may include the location of the detecting device 200, the remaining power of the power source 202, the distance to one or more charging stations, the distance to one or more base stations, the locations of other detecting devices, the types of the other detecting devices, the remaining capacity of the syringe containers 240, the likely locations of additional, uncollected syringes, the sensor data, context data, the characteristics of syringes in the syringe containers 240, the locations of identified, uncollected syringes, and/or any other similar factor. The factors may also include environmental conditions such as locations of any nearby people or animals. In some implementations, the base stations and the charging stations may be the same locations, which may be locations where the detecting device 200 can charge the power source 202 and empty the syringe containers 240. The actions may include moving to a charging station, moving to assist one or more detecting devices, becoming immobile and protect an uncollected syringe, moving to the likely location of an additional syringe, and/or any other similar action.

The processors 210 may utilize various rules to determine the subsequent action for the detecting device 200. For example, the processors 210 may determine to instruct the flight control hardware 206 to move the detecting device 200 to the nearest likely location of an uncollected syringe if the power source 202 is above fifty percent, the syringe containers 240 each have remaining capacity, and none of the collected syringes have a risk score of above 0.9. As another example, the processors 210 may determine to instruct the flight control hardware 206 to move the detecting device 200 to the nearest base station if any of the syringe containers 240 are full. As another example, the processors 210 may determine to instruct the flight control hardware 206 to cause the detecting device 200 to be disabled and protect an uncollected syringe if the risk score of the uncollected syringe is above 0.6 and there is not another detecting device that can likely collect the syringe within a threshold period of time, such as ten minutes. As another example, the processors 210 may determine to instruct the flight control hardware 206 to cause the detecting device 200 to travel to the nearest base station to deposit the syringes if the syringe containers 240 are storing a syringe that has a risk score of above 0.9.

In some implementations, the syringe identifier 238, or any subcomponent of the syringe identifier 238, may be configured to determine a confidence score that its output is correct. For example, the confidence score may be on a range from zero to one, where zero indicates no confidence and one indicates one hundred percent confidence. A confidence score of 0.5 may indicate an equal chance that the output is correct. In some implementations, if the confidence score is within a range, such as 0.4 to 0.6, then the syringe identifier 238 or other component may capture an image of the area in question and transmit that image and/or video to another computing device for further processing. The further processing may include a user viewing the image and/or video and inputting whether the image includes a syringe, the type of syringe, the integrity of the syringe, and/or the risk of the syringe.

In some implementations, the syringe identifier 238 may determine the distance and size of syringes and other objects from the detecting device 200. The syringe identifier can translate the distance and size of the syringes and other objects into GPS coordinates using various methods, such as Simultaneous Location and Mapping (SLAM).

In some implementations, the syringe identifier 238 may use the sensor data, for example LIDAR data, to determine a map of the environment where the detecting device 200 is analyzing. The map may be a three-dimensional map, and the syringe identifier 238 may populate the map with likely locations of syringes. The syringe identifier 238 may include a confidence score that the identified locations include a syringe. The map may be used by the detecting device 200 to collect the syringes and/or provided to another computing device for collecting by another device and/or for manual collection.

In addition to local processors, or alternatively, the detecting device 200 may utilize cloud-based processing and/or memory. In some implementations, syringe identifier 238, the syringe type determiner 246, the syringe integrity determiner 248, and/or the syringe risk determiner 250 may be implemented using cloud-based processing. In this case, the syringe identifier 238, the syringe type determiner 246, the syringe integrity determiner 248, and/or the syringe risk determiner 250 may utilize the models 232 and/or rules 234 using cloud-based processing. In some implementations, the detecting device 200 may transmit sensor data to the cloud for further analysis. Processing of the sensor data may occur in the cloud and the detecting device 200 may receive data indicating a likelihood that the detecting device 200 is in the vicinity of a syringe, a likely type of the syringe, a likely integrity of the syringe, and/or a likely risk posed by the syringe.

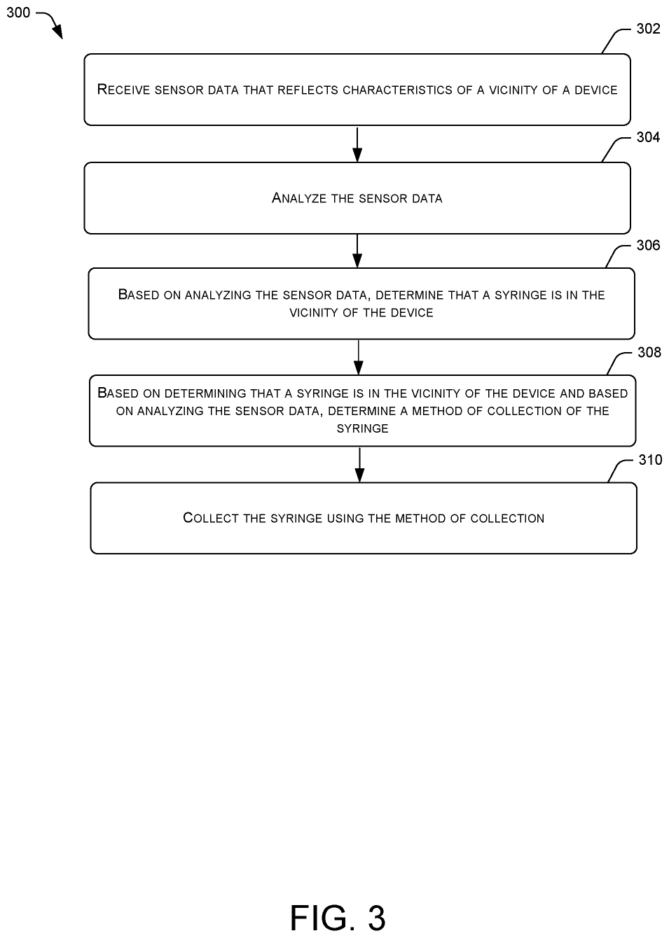

FIGS. 3 through 5 present processes 300, 400, and 500 that relate to operations of the WMOC server 130 and/or the detecting device 140 or 200. Each of the processes 300, 400, and 500 illustrate a collection of blocks in a logical flow chart, which represents a sequence of operations that can be implemented in hardware, software, or a combination thereof. In the context of software, the blocks represent computer-executable instructions that, when executed by one or more processors, perform the recited operations. Generally, computer-executable instructions may include routines, programs, objects, components, data structures, and the like that perform particular functions or implement particular abstract data types. The order in which the operations are described is not intended to be construed as a limitation, and any number of the described blocks can be combined in any order and/or in parallel to implement the process. For discussion purposes, the processes 300 and 400 are described with reference to the network server environment 100 of FIG. 1 and or the detecting device 200 of FIG. 2.

FIG. 3 illustrates a process 300 for identifying and collecting a syringe. The process 300 collects and analyzes sensor data collected and/or received by a detecting device 200. Based on the sensor data, the detecting device 200 determines the location of a syringe and a method to collect the syringe. The process 300 will be described from the perspective of the detecting device 200, which can interact with the WMOC server 130 and additional detecting devices to receive various types of data that may be different than sensor data generated by the detecting device 200.

At block 302, the detecting device 200 receives sensor data that reflects the characteristics of a vicinity of the detecting device 200. The detecting device 200 may include various sensor such as a camera, a LIDAR, image sensor, shape detector, metal detector, optical sensor, chemical sensor, UV sensor, proximity sensor, light sensor, IR sensor, accelerometer, temperature sensor, an altitude sensor, a global positioning system (GPS) sensor, control setting sensors, propulsion setting sensors, and the like. The detecting device 200 may also receive sensor data from nearby detecting devices. The detecting device 200 may also receive context data from a server. The context data may include data identifying an area where the detecting device 200 is located, current events, current weather, and/or any other similar context information.

At block 304, the detecting device 200 analyzes the sensor data. The detecting device 200 may use various models and/or rules to analyze the sensor data and/or the context data. In some implementations, the detecting device 200 may provide the sensor data to the WMOC server 130 for analysis. The WMOC server 130 may analyze the sensor data in a similar manner such as using models and/or rules. The models may be trained using machine learning and historical data. The rules may be user-defined and/or may be generated based on the historical data. The detecting device 200 may select a model and/or rule based on the type of sensor data and context data. Different models and/or rules may be configured to analyze different types of data.

At block 306, the detecting device 200 determines that a syringe is in the vicinity of the detecting device 200 based on analyzing the sensor data. The models and/or rules may output data indicating a likelihood that a syringe is in the vicinity of the detecting device.

At block 308, the detecting device 200 determines a method of collection of the syringe based on determining that a syringe is in the vicinity of the detecting device 200 and based on analyzing the sensor data. The detecting device 200 may use additional models and/or rules to determine the type of syringe, the integrity of the syringe, and/or the risk posed by the syringe. The additional models and/or rules may analyze the sensor data, the context data, and/or the likely location of the syringe. Based on the type of syringe, the integrity of the syringe, and/or the risk posed by the syringe, the detecting device 200 may a method of collection of the syringe. The method of collection may include an implement to use to collect the syringe and a location to store the syringe.

At block 310, the detecting device 200 collects the syringe using the method of collection. The detecting device 200 may attempt to collect the syringe and store the syringe using the determined method and in the storage location, respectively. The detecting device 200 may attempt to use an additional method of collection if the initial method of collection is unsuccessful.

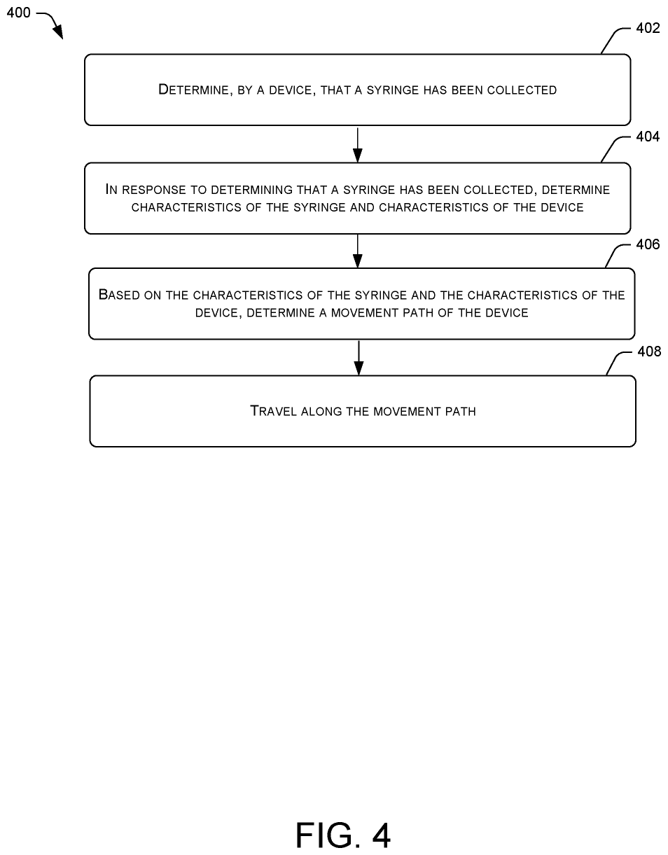

FIG. 4 illustrates a process 400 for determining a movement path for a detecting device 200 after collection of a syringe. The process 400 determines various characteristics of the syringe, the environment around the detecting device 200, the context of the vicinity of the detecting device 200, and/or any other similar data. Based on analyzing this data, the process 400 determines a movement path for the detecting device 200. The process 300 will be described from the perspective of the detecting device 200, which can interact with the WMOC server 130 and additional detecting devices to receive various types of data that may be different than sensor data generated by the detecting device 200.

At block 402, the detecting device 200 determines that the detecting device 200 has collected a syringe. The detecting device 200 may make this determination based on sensor data. Based on analyzing the sensor data, the detecting device 200 may determine that a syringe is no longer in the vicinity of the detecting device 200. Additionally, or alternatively, the detecting device 200 may make this determination based on detecting a decrease in the remaining capacity of the syringe containers 240.

At block 404, the detecting device 200 determines characteristics of the syringe and characteristics of the detecting device 200 in response to determining that the detecting device 200 has collected a syringe. The characteristics of the syringe may include the type of syringe, the integrity of the syringe, and/or the risk posed by the syringe. The detecting device 200 may have previously determined these characteristics in preparation to collect the syringe. In some implementations, the detecting device 200 may also analyze sensor data, context data, additional data received from a server, additional data received from other detecting devices, and/or any other similar data.

At block 406, the detecting device 200 determines a movement path of the detecting device 200, based on the characteristics of the syringe and the characteristics of the detecting device 200. The characteristics of the detecting device 200 may include the sensor data generated by the sensors of the detecting device and/or the context of the detecting device 200. The context data may include locations of nearby detecting devices, location of base and/or charging stations, data identifying an area of the detecting device 200, and/or any other similar data. The movement path may include moving to the likely location of an additional syringe, moving to a charging station, moving to assist one or more detecting devices, becoming immobile and protecting an uncollected syringe, depositing the syringes at a base station or other location, and/or any other similar action.

In some implementations, the movement path may be predetermined. In this case, the movement path may not be based on the characteristics of the syringe and the characteristics of the detecting device 200. The movement path may be predetermined based on an area for the detecting device 200 to analyze for possible syringes. For example, the movement path may include the various portions of a playground.

At block 408, the detecting device 200 travels along the movement path. The detecting device 200 may activate the flight control hardware 206 and the propulsion hardware 204 to move along the path and perform any corresponding actions.

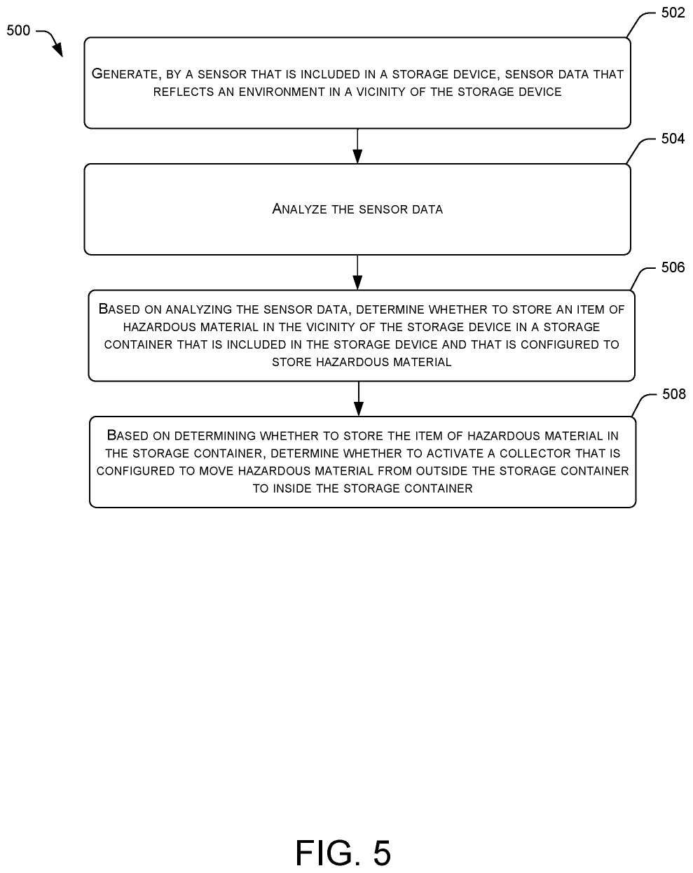

FIG. 5 illustrates a process 500 for determining whether to collect hazardous material using a storage device. The process 500 generates sensor data. Based on that sensor data, the process 500 determines whether to store hazardous material in a storage container of the storage device. The process 500 may activate a collector to automatically move the hazardous material into the storage container. The process 500 will be described from the perspective of the detecting device 200, which may be referred to as a storage device that is configured to store hazardous material. In some implementations, the process 500 may be performed by one or more devices illustrated in FIG. 1 or 2. In some implementations, the process 500 may be performed by one or more virtual machines.

At block 502, the storage device includes a sensor that generates sensor data that reflects an environment in a vicinity of the storage device. In some implementations, the sensor includes one or more of a camera, a light detection and ranging device, an RFID detector, a metal detector, a chemical detector, a fluorescence detector, a microphone, a thermometer, a light sensor, a proximity sensor, a pressure sensor, an accelerometer, a gyroscope, a gravity sensor, a magnetometer, a humidity sensor, and/or a barometer. The sensor may be included in the storage device and/or located on another device that the user of the storage device may be carrying around. In some implementations, the sensors may be fixed such as to a lamp post, a wall, and/or any other fixed object. In some implementations, the sensor is an RFID reader and the sensor data is the unique identifier stored in an RFID tag detected by the RFID reader.

At block 504, the storage device analyzes the sensor data. In some implementations, the storage device includes a processor that is configured to analyze the sensor data. The storage device may use various models trained using machine learning and/or rules to analyze the sensor data.

Based on analyzing the sensor data and at block 506, the storage device determines whether to store an item of hazardous material in the vicinity of the storage device in a storage container that is included in the storage device and that is configured to store hazardous material. In some implementations, the hazardous material is a syringe. In some implementations, the storage device determines an integrity of the item of hazardous material based on analyzing the sensor data. The integrity may reflect whether the hazardous material is broken or otherwise not usable for its intended purpose, for example a syringe with a broken needle or damaged plunger. In some implementations, the determines a risk of the item of hazardous material based on analyzing the sensor data. The risk may reflect the likelihood that the hazardous material poses a risk to a person. For example, a syringe may pose a risk because of the needle.

In some implementations, the storage device may determine characteristics of the item of hazardous material. The characteristics may be accessible through a database or other type of organizational tool. The database may include the unique identifier and other characteristics of the hazardous material. The characteristics of the hazardous material may include a distribution location, a current location, measurements, and/or a type of the hazardous material. For syringes, the characteristic may include the syringe's specifications, such as type, needle gauge, needle length, etc. In some implementations, the characteristics of the hazardous material may be stored in the database at the time the hazardous material is distributed. For example, at the time a syringe is distributed, an RFID reader may scan the RFID tag and the characteristics of the syringe may be stored in the database along with the unique identifier of the RFID tag. In some implementations, at the time a syringe is manufactured, an RFID tag may be applied to the syringe and the characteristics of the syringe may be stored in the database along with the unique identifier of the RFID tag.

Based on determining whether to store the item of hazardous material in the storage container and at block 508, the storage device determines whether to activate a collector that is configured to move hazardous material from outside the storage container to inside the storage container. In some implementations, the storage device determines to store the hazardous material in the storage container and determines to activate the collector. In some implementations, the storage device determines to bypass storing the hazardous material in the storage container and bypasses activating the collector. In some implementations, the storage device determines to store the hazardous material in another storage container included in the storage device and activates a collected to move the hazardous material to the other storage container. The storage device may select a storage container based on a type of hazardous material, the capacity of the various storage containers included in the storage device, and/or any other similar feature. If the storage device bypasses storage of the hazardous material, the storage device may output a notification indicating that the reason for not storing the hazardous material. Some example reasons for not storing the hazardous material may include the storage container is full, the hazardous material is more dangerous in the storage container than at its present location, and/or any other similar reason.

In some implementations, the storage device may be configured to determine the unique identifier of the hazardous material upon storage of the hazardous material. For example, the collection device may include an RFID reader that scans RFID tags. In some implementations, the storage device is any type of device that is configured to perform an automatic action before, during, and/or after collection of the hazardous material. For example, the storage device may be a scooping device that includes a motor to activate a sweeping component to move the hazardous material into the storage container of the collection device. The motor may respond to the control instruction. As another example, the storage device may be a device container that is placed over the hazardous material. The storage container of the storage device may include an automatic door that opens and closes from the power of a motor that responds to the control instruction. A user may place the collection device over the hazardous material. The motor may receive the control instruction, and the automatic door opens and closes to collect and store the hazardous material.

Although the subject matter has been described in language specific to features and methodological acts, it is to be understood that the subject matter defined in the appended claims is not necessarily limited to the specific features or acts described herein. Rather, the specific features and acts are disclosed as exemplary forms of implementing the claims.

What is claimed is:

1. A storage device comprising:
a storage container that is configured to store hazardous material;
a collector that is configured to move hazardous material from outside the storage container to inside the storage container;
a sensor that is configured to generate sensor data that reflects a characteristic of an environment in a vicinity of the storage device; and
a processor that is configured to:
analyze the sensor data;
based on analyzing the sensor data, determine whether to store the item of hazardous material in the storage container; and
based on determining whether to store the item of hazardous material in the storage container, determine whether to activate the collector to move the item of hazardous material from outside the storage container to inside the storage container.

2. The storage device of claim 1, wherein:
the sensor is an RFID reader,
the sensor data is unique identifier stored in an RFID tag on the item of hazardous material.

3. The storage device of claim 1, wherein:
determining whether to store the item of hazardous material in the storage container comprises determining to store the item of hazardous material in the storage container, and
determining whether to activate the collector to move the item of hazardous material from outside the storage container to inside the storage container comprises determine to activate the collector to move the item of hazardous material from outside the storage container to inside the storage container.

4. The storage device of claim 1, wherein:
determining whether to store the item of hazardous material in the storage container comprises determining to bypass storing the item of hazardous material in the storage container,
determining whether to activate the collector to move the item of hazardous material from outside the storage container to inside the storage container comprises determine to bypass activating the collector to move the item of hazardous material from outside the storage container to inside the storage container, and
the processor is configured to:
based on determining to bypass storing the item of hazardous material in the storage container, generate and output a notification indicating that the storage device is bypass storing the item of hazardous material in the storage container.

5. The storage device of claim 1, wherein the processor is configured to:
determine characteristics of the item of hazardous material,
wherein determining whether to store the item of hazardous material in the storage container is further based on the characteristics of the item of hazardous material.

6. The storage device of claim 5, wherein the characteristics of the item of hazardous material includes a distribution location, a current location, measurements, and a type of the hazardous material.

7. The storage device of claim 1, wherein the sensor comprises a camera, a light detection and ranging device, an RFID detector, a metal detector, a chemical detector, a fluorescence detector, a microphone, a thermometer, a light sensor, a proximity sensor, a pressure sensor, an accelerometer, a gyroscope, a gravity sensor, a magnetometer, a humidity sensor, and a barometer.

8. The storage device of claim 1, wherein the item of hazardous material is a syringe.

9. The storage device of claim 1, comprising:
an additional storage container that is configured to store hazardous material,
wherein the processor is configured to determine whether to store the item of hazardous material in the additional storage container based on analyzing the sensor data.

10. The storage device of claim 1, wherein the processor is configured to:
based on analyzing the sensor data, determine an integrity of the item of hazardous material,
wherein determining whether to store the item of hazardous material in the storage container is based on the integrity of the item of hazardous material.

11. The storage device of claim 1, wherein the processor is configured to:
based on analyzing the sensor data, determine a risk associated with the item of hazardous material,
wherein determining whether to store the item of hazardous material in the storage container is based on the risk associated with the item of hazardous material.

12. A method comprising:
generating, by a sensor that is included in a storage device, sensor data that reflects an environment in a vicinity of the storage device;
analyzing the sensor data;
based on analyzing the sensor data, determining whether to store an item of hazardous material in the vicinity of the storage device in a storage container that is included in the storage device and that is configured to store hazardous material; and
based on determining whether to store the item of hazardous material in the storage container, determining whether to activate a collector that is configured to move hazardous material from outside the storage container to inside the storage container.

13. The method of claim 12, wherein:
the sensor is an RFID reader,
the sensor data is unique identifier stored in an RFID tag on the item of hazardous material.

14. The method of claim 12, wherein:
determining whether to store the item of hazardous material in the storage container comprises determining to store the item of hazardous material in the storage container, and
determining whether to activate the collector to move the item of hazardous material from outside the storage container to inside the storage container comprises determine to activate the collector to move the item of hazardous material from outside the storage container to inside the storage container.

15. The method of claim 12, wherein:
determining whether to store the item of hazardous material in the storage container comprises determining to bypass storing the item of hazardous material in the storage container,
determining whether to activate the collector to move the item of hazardous material from outside the storage container to inside the storage container comprises determine to bypass activating the collector to move the item of hazardous material from outside the storage container to inside the storage container, and
the method comprises:
based on determining to bypass storing the item of hazardous material in the storage container, generating and output a notification indicating that the storage device is bypass storing the item of hazardous material in the storage container.

16. The method of claim 12, comprising:
determining characteristics of the item of hazardous material,
wherein determining whether to store the item of hazardous material in the storage container is further based on the characteristics of the item of hazardous material, and
wherein the characteristics of the item of hazardous material includes a distribution location, a current location, measurements, and a type of the hazardous material.

17. The method of claim 12, wherein the sensor comprises a camera, a light detection and ranging device, an RFID detector, a metal detector, a chemical detector, a fluorescence detector, a microphone, a thermometer, a light sensor, a proximity sensor, a pressure sensor, an accelerometer, a gyroscope, a gravity sensor, a magnetometer, a humidity sensor, and a barometer.

18. The method of claim 12, wherein the item of hazardous material is a syringe.

19. The method of claim 12, comprising:
determining whether to store the item of hazardous material in an additional storage container that is included in the storage device based on analyzing the sensor data.

20. The method of claim 12, comprising:
based on analyzing the sensor data, determining an integrity of the item of hazardous material and a risk associated with the item of hazardous material,
wherein determining whether to store the item of hazardous material in the storage container is based on the integrity of the item of hazardous material and the risk associated with the item of hazardous material.
